(19) United States
(12) Patent Application Publication
(10) Pub. No.: US 2007/0043267 A1
(43) Pub. Date: Feb. 22, 2007

(54) Neuropsychological assessment of alternate perspectives and associated methods

(76) Inventors: Sarah Leech Kemp, Tulsa, OK (US); Marit Korkman, Helsinki (FI)

Correspondence Address: JACQUELINE E. HARTT, PH.D;ALLEN, DYER, DOPPELT, MILBRATH & GILCHRIST, P.A., P.O. BOX 3791, ORLANDO, FL, 32802-3791, US

(21) Appl. No.: 11/206,520

(22) Filed: Aug. 18, 2005

(51) Int. Cl. A61B 5/00

(52) U.S. Cl. 600/300

(57) ABSTRACT

A neuropsychological assessment of perception of emotional expression in other people tests an ability to understand mental functions such as belief, intention, deception, emotion, imagination, and pretending. In a particular embodiment, a first part assesses the ability to understand that others have their own thoughts, ideas, and feelings that may be different from his/her own. The subject is read various scenarios or shown pictures, and is then asked questions that require knowledge of another individual's point of view to answer correctly. A second part assesses the subject's ability to recognize the appropriate affect given various social contexts. The subject is shown a picture depicting a social context and is asked to select a photograph from four options that depicts the appropriate affect for the target person in the picture. The assessment offers a reliable indicator of a subject's ability to comprehend the motivations and perceptions of other individuals.

### BACKGROUND OF THE INVENTION

1. Field of the Invention

The present invention relates to neuropsychological assessment systems and methods, and, more particularly, to such systems and methods for assessing a subject's understanding of mental functions for use, for example, in diagnosing autism disorders.

2. Description of Related Art

Difficulty in understanding other perspectives has been recognized as a core feature of autism spectrum disorders, although impairment in this area is at present not part of the DSM-IV criteria for diagnosis of these disorders.

Theory of Mind tasks have been proposed previously, but there is no known standardized assessment extant for use within a broader pediatric neuropsychological assessment. Further, there is no known assessment designed for diagnosing very young children.

### SUMMARY OF THE INVENTION

The present invention is directed to a neuropsychological assessment of a subject's perception of emotional expression in other people. The assessment tests a subject's ability to understand mental functions such as belief, intention, deception, emotion, imagination, and pretending.

In a particular embodiment, the assessment comprises two parts. A first part assesses the subject's ability to understand that others have their own thoughts, ideas, and feelings that may be different from his/her own. The subject is read various scenarios or shown pictures, and is then asked questions that require knowledge of another individual's point of view to answer correctly.

A second part assesses the subject's ability to recognize the appropriate affect given various social contexts. The subject is shown a picture depicting a social context and is asked to select a photograph from four options that depicts the appropriate affect for the target person in the picture.

The assessment of the present invention offers a reliable indicator of a subject's ability to comprehend the motivations and perceptions of other individuals. A deficit in this area is frequently seen in children on the autistic spectrum.

In scoring the assessment of the present invention, each correct item is scored with one to two points, with a decile score less than or equal to approximately 3 being indicative of a presence of an autistic disorder. It is to be understood that this decile figure may be used in a particular embodiment, and that other embodiments may select a different decision point.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWINGS

FIGS. 1-5 are exemplary stimulus pictures and sets of possible answers.

FIG. 6 is an exemplary stimulus page, having a drawing of a scene and a set of possible photos depicting an emotional state.

### DETAILED DESCRIPTION OF THE PREFERRED EMBODIMENTS

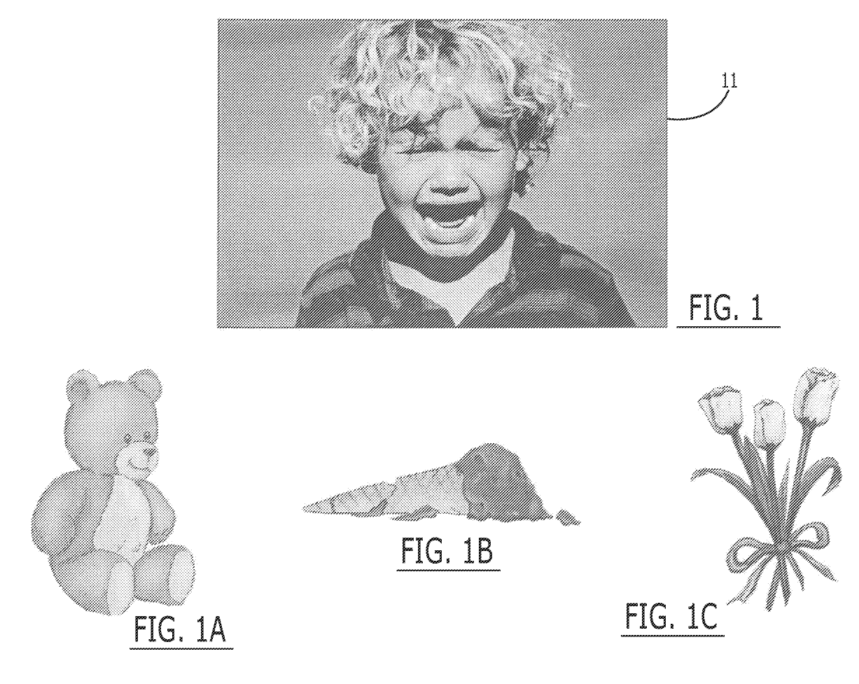

A description of the preferred embodiments of the present invention will now be presented with reference to FIGS. 1-6.

The assessment of the present invention includes a plurality of administration instructions and display test pages. In a typical mode of administration, the test is provided bound in a spiral book, with the pages arranged so that the administration instructions are facing the test administrator and the test pages are facing the subject. For the first set of items, the book will typically be in a tented, or upright, position.

An exemplary embodiment of the assessment of the present invention includes a plurality of items that are administered orally to the subject. The subject is asked to listen closely to the administrator and to look carefully at any pictures that are displayed. In the first part of the assessment, first, the administrator performs an incongruous act. For example, the administrator may point at his/her shoe and say “Look, this is my hat!” and then ask “Why did I say that?” In a second item, the administrator places an object in one of his/her hands in view of the subject and then closes both hands. The subject is then asked to point to the hand holding the object. This may be carried out several times.

For the next set of items, the stimulus book will typically be laid flat so that the administrator can see where the subject is pointing. The next item includes displaying the page of FIG. 1 and asking “Show me what made the boy cry,” referring to the top photograph 11. The subject then selects among choices A-C, wherein a dropped ice cream cone (B) should be perceived as reason for crying.

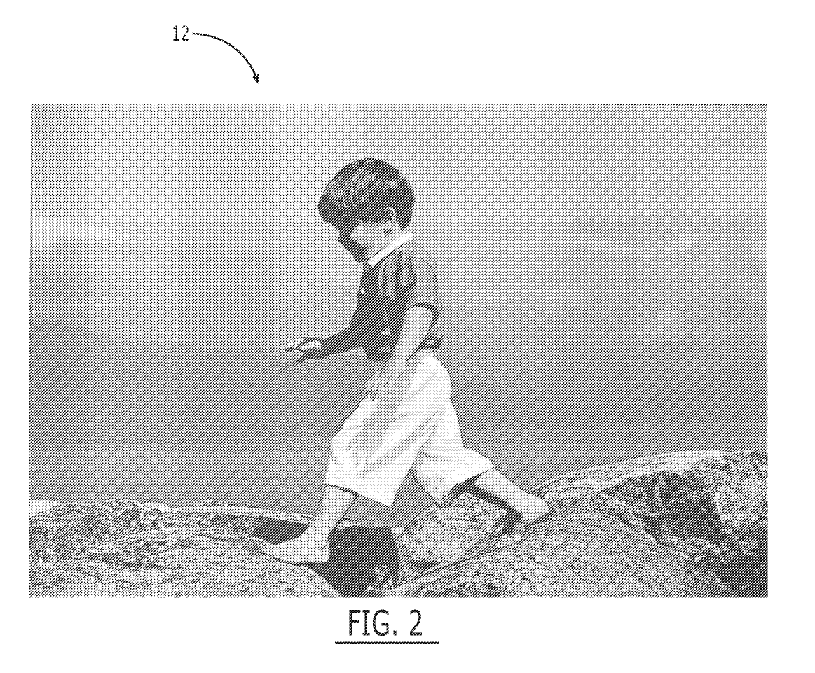

In the photograph 12 of FIG. 2, a boy is shown walking on rocks. The administrator says “Now I am going to act out a rhyme. Watch and do what I do. Running on the rocks is fun.” (The examiner is instructed to “run” two fingers across the table, gesturing for the examinee to imitate.) “When we run across the rocks, we come home with stinky socks.” (Examiner pinches nose and then resumes running fingers across the table.) “When we run across the rocks.”

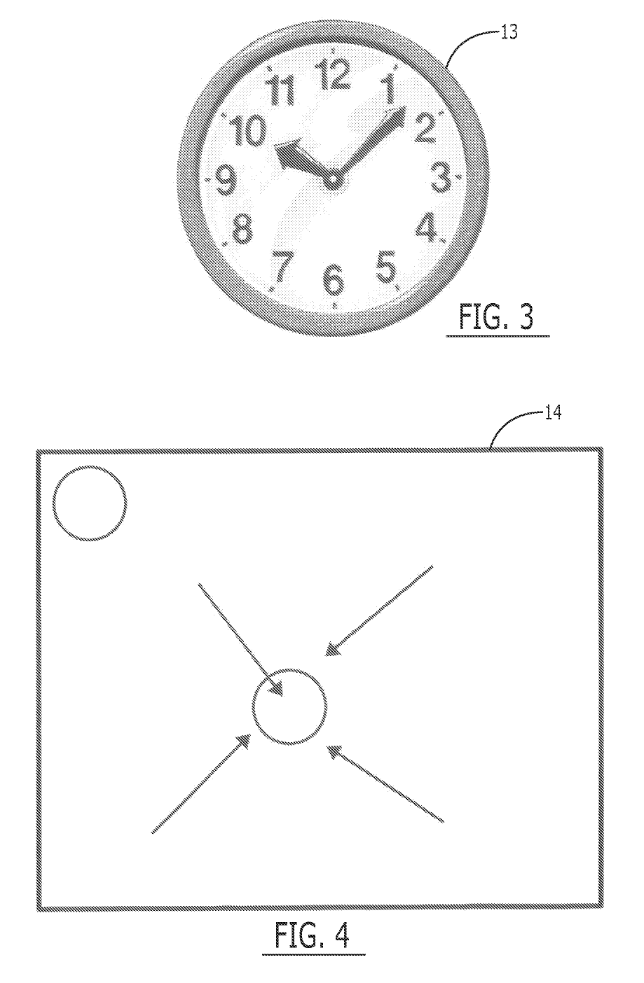

When the clock 13 of FIG. 3 is shown, the subject is read: “The hands on the clock are near 10 o'clock. Show me the hands on the clock.”

When shown the picture 14 of FIG. 4, the subject is asked, when the administrator points to the circle in the center, “How does this circle feel?” When pointing to the circle in the upper left-hand corner, the administrator then asks “How does this circle feel?” Then, “How do the arrows feel?”

Items may also be given that require the subject to listen to a story and provide a rationale for an action taken by one of the characters in the story.

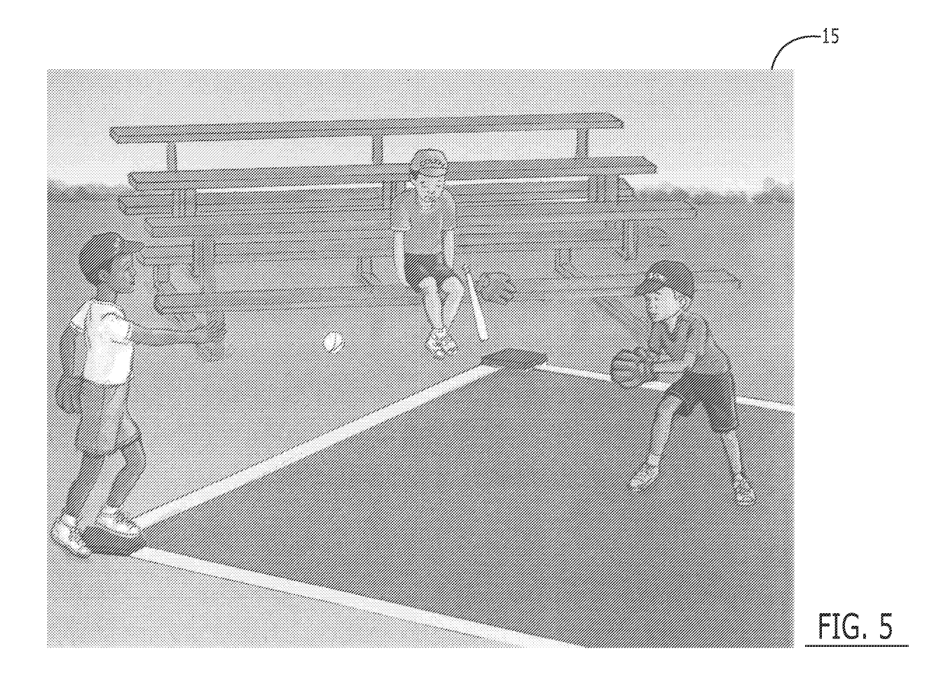

When the picture 15 of FIG. 5 is displayed, the administrator points to the two boys playing together and says: “Lee is Joe's best friend. They always play together. Colin would like to play with Joe, but Joe is always with Lee, so he doesn't play with Colin.” The administrator points to the boy on the bleachers. “One day, Lee was sick and had to stay home. That day Joe played with Colin. How did that make Lee feel? How did Colin feel?”

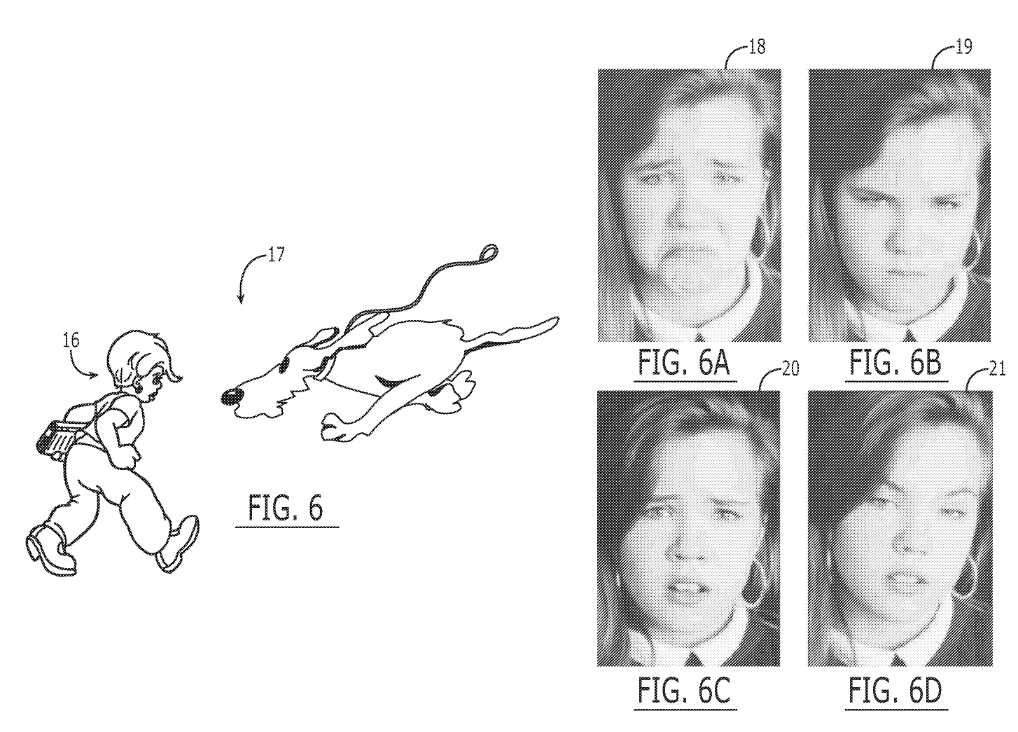

In the second part of the assessment, the examiner displays illustrations such as that in FIG. 6 and says: “Here is Julia,” pointing to the girl 16 in the drawing 17, “and here are four pictures of Julia,” pointing to the photos A-D 18-21. “Point to the picture that shows how Julia feels over here,” pointing to the drawing 17.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

What is claimed is:

1. A method of assessing a subject for an autistic disorder comprising the steps of:
administering a first subtest comprising:
communicating a scenario to a subject containing a first individual in a predefined situation; and
asking the subject for a response to a question relating to a perspective of the first individual;
administering a second subtest comprising:
displaying to the subject a representation of a second individual in a social context; and
displaying to the subject a plurality of depictions of the second individual, wherein in each depiction the second individual is exhibiting a different emotion, one of the depictions displaying an appropriate emotion responsive to the social context;
eliciting a response from the subject comprising a selection from among the depictions;
scoring the subject responses to the first and the second subtest; and
determining from the scoring step an indication of a presence of an autistic disorder.

2. The method recited in claim 1, wherein the communicating step comprises one of reading a story to the subject and displaying a picture to the subject.

3. The method recited in claim 1, wherein the plurality of depictions comprise pictures of a face of the second individual.

4. The method recited in claim 1, wherein the response-eliciting step includes asking the subject about feelings of the second individual in the social context.

5. The method recited in claim 1, further comprising the step of administering a third test comprising:
performing an act and making a statement that is incongruous with the act; and
asking a question of the subject regarding the incongruity.

6. The method recited in claim 1, further comprising the step of administering a fourth test comprising:
placing an object in one hand in view of the subject; and
asking the subject to indicate in which hand the object is.

7. An assessment of a subject for an autistic disorder comprising:
a first subtest comprising a scenario containing a first individual in a predefined situation;
means for communicating the scenario to a subject;
means for asking the subject for a response to a question relating to a perspective of the first individual;
a second subtest comprising a representation of a second individual in a social context; and
means for displaying to the subject a plurality of depictions of the second individual, wherein in each depiction the second individual is exhibiting a different emotion, one of the depictions displaying an appropriate emotion responsive to the social context;
means for eliciting a response from the subject comprising a selection from among the depictions;
means for scoring the subject responses to the first and the second subtest; and
means for determining from the scoring step an indication of a presence of an autistic disorder.

8. The assessment recited in claim 7, wherein the communicating means comprises one of means for reading a story to the subject and means for displaying a picture to the subject.

9. The assessment recited in claim 7, wherein the plurality of depictions comprise pictures of a face of the second individual.

10. The assessment recited in claim 7, wherein the response-eliciting means includes means for asking the subject about feelings of the second individual in the social context.

11. The assessment recited in claim 7, further comprising a processor and software resident on the processor, and wherein the communicating and displaying means comprise an output device in signal communication with the processor, the software adapted to direct the output device to communicate the scenario and to display the depictions.

12. The assessment recited in claim 7, further comprising a third test comprising:
means for performing an act and making a statement that is incongruous with the act; and
means for eliciting a response from the subject regarding the incongruity.

13. The assessment recited in Claim. 7, further comprising a fourth test comprising:
an object placeable in one hand in view of the subject; and
means for eliciting from the subject an indication of in which hand the object is.
